(19) United States
(12) Patent Application Publication
(10) Pub. No.: US 2017/0312469 A1
(43) Pub. Date: Nov. 2, 2017

(54) CONTINUOUS POSITIVE AIR PRESSURE MASK SYSTEM

(71) Applicant: Gary W. Lay, Galesburg, IL (US)

(72) Inventor: Gary W. Lay, Galesburg, IL (US)

(21) Appl. No.: 15/208,146

(22) Filed: Jul. 12, 2016

Related U.S. Application Data
(60) Provisional application No. 62/196,950, filed on Jul. 25, 2015.

(51) Int. Cl. A61M 16/06; A61M 16/00; A61M 16/08

(52) U.S. Cl. CPC A61M 16/0683; A61M 16/0875; A61M 16/0057; A61M 2210/0625; A61M 2210/0618

(57) ABSTRACT

A continuous positive air pressure mask system, configured to fit around a human head, including a mask, an air inlet distribution chamber a first and second air channel and head gear. The mask configured is to fit over at least one of a nose and a mouth on the head. The air inlet distribution chamber includes an air inlet chamber and a chamber air inlet configured to connect to a continuous positive air pressure machine to receive pressurized air. The air inlet chamber includes a bottom surface contoured to fit the top of the head. The first and second air channels fluidly connect the mask to the air distribution chamber. The head gear releasably attaches to the mask to hold the mask over the at least one of a nose and a mouth.

### PRIORITY

This application claims priority to and incorporates by reference in its' entirety, U.S. Provisional Patent Application No. 62/196,950, entitled “Continuous Positive Air Pressure Mask System”, and filed Jul. 25, 2015.

### BACKGROUND OF THE INVENTION

The present invention generally relates to masks for use with a continuous positive air pressure (CPAP) system.

The Comfort Fit 360 was designed to alleviate most of the problems with current CPAP masks and devices.

This continuous positive air pressure is the bases of for devices meant to help sleep apnea patients. In reality most users of these devices cannot use them the entire night because of several problems. If people could stay in the same position all night long most devices would work better. But people don't, normally changing their sleeping position from several to dozens of times causing problems to occur with the CPAP mask and system.

The first and most common problem may be that a person rolls over, and the mask becomes dislodged breaking the air tight seal. There is no longer a continuous positive air pressure.

The second problem happens when a person rolls over and gets in tangled in the air hoses or the air hoses get disconnected.

### BRIEF DESCRIPTION OF THE DRAWINGS

FIG. 1 is a front view of a CPAP mask system according to an exemplary embodiment of the invention.

FIG. 2 is a front view of tubing for the CPAP mask system according to an exemplary embodiment of the invention.

FIG. 3 is a front view of the CPAP mask according to an exemplary embodiment of the invention.

FIG. 4 is a back view of the CPAP mask according to an exemplary embodiment of the invention.

FIG. 5 is a front view of air channels for the CPAP mask system according to an exemplary embodiment of the invention.

FIG. 6 is a side view of air channels for the CPAP mask system according to an exemplary embodiment of the invention.

FIG. 7 is a perspective view of headgear for the CPAP system according to an exemplary embodiment of the invention.

FIG. 8 is a top view of an air inlet distribution chamber for the CPAP mask system according to an exemplary embodiment of the invention.

FIG. 9 is a front side view of an air inlet distribution chamber for the CPAP mask system according to an exemplary embodiment of the invention.

FIG. 10 is a front view of a human head wearing the CPAP mask system according to an exemplary embodiment of the invention.

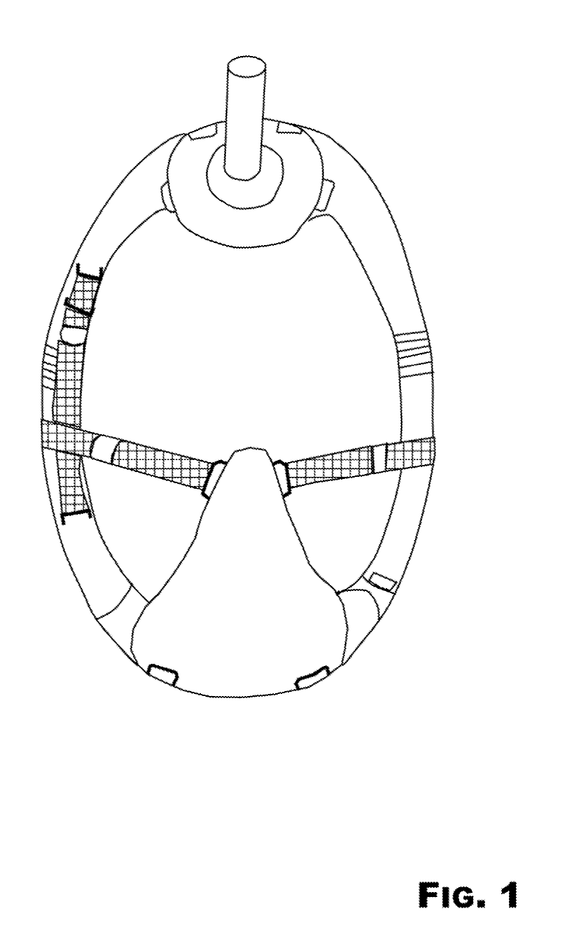

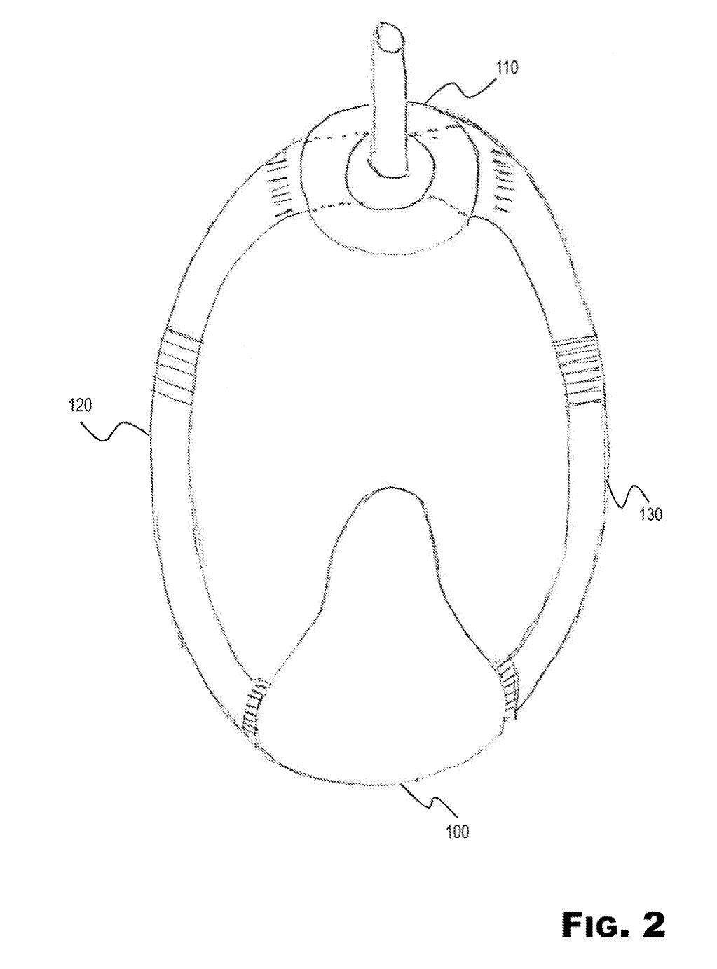

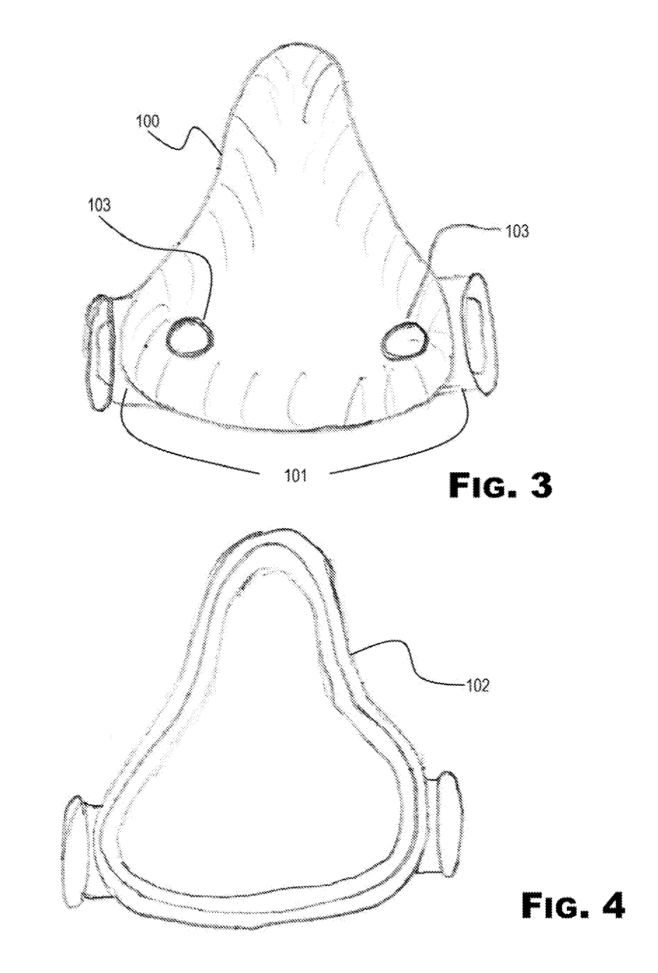

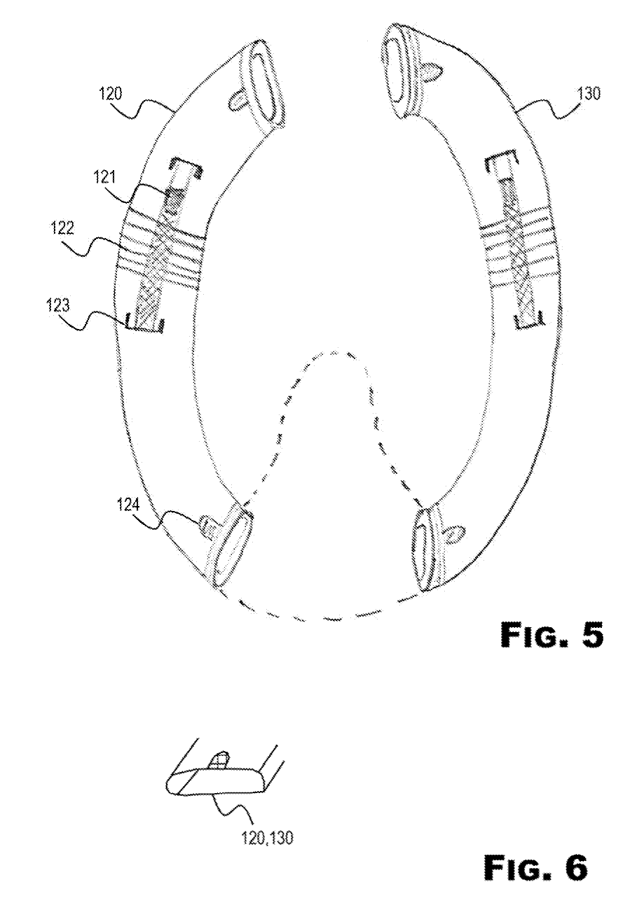

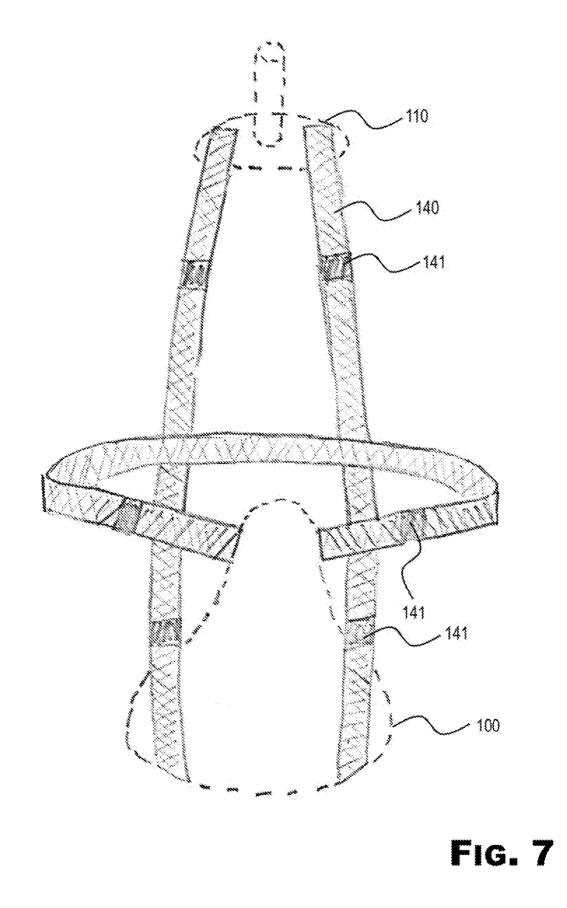

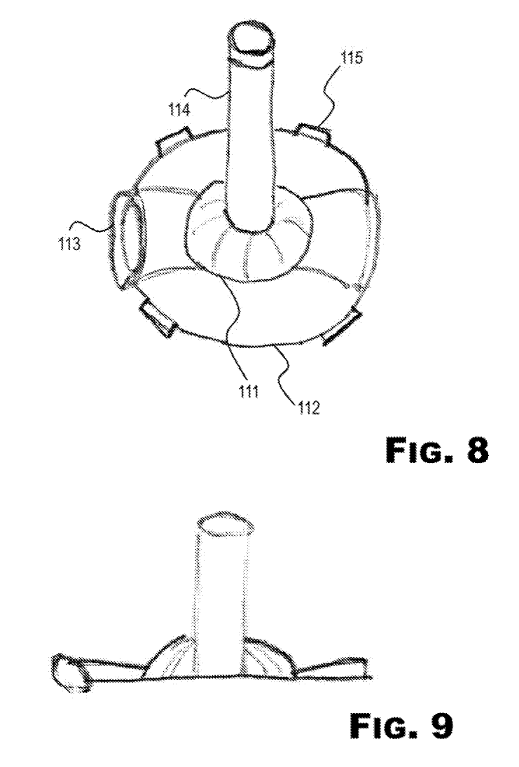

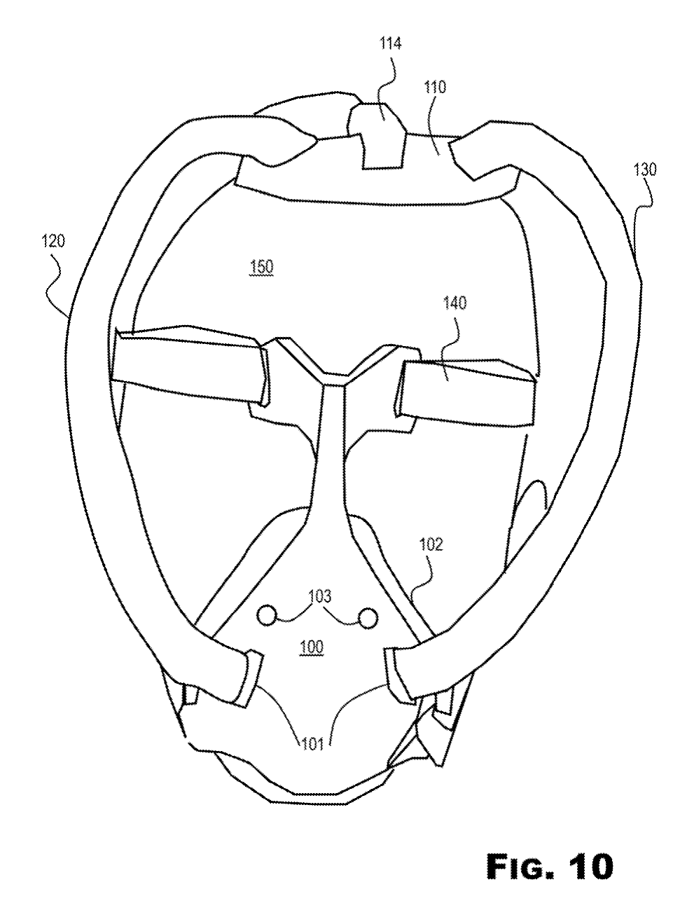

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

The illustrations in the drawings provide views of how the CPAP mask system works. The following is a listing of element numbers and the components they identify.

| Element Number | Component |
|---|---|
| 100 | Full CPAP Mask |
| 101 | Side Flat Channel Air Inlet |
| 102 | Double Flexible Air Inlet Seals |
| 103 | Exhale Valves |
| 110 | Air Inlet Distribution Chamber |
| 111 | Air Inlet Chamber |
| 112 | Comfort Fit Out Cap |
| 113 | Low Profile Air Inlet |
| 114 | 360° Snap Fit Air Inlet |
| 115 | Head Gear Connectors |
| 120 | First Low Profile Air Chamber |
| 130 | Second Low Profile Air Chamber |
| 121 | Adjustable Length Straps |
| 122 | Adjustable Length Channels |
| 123 | Tie Hooks for Straps |
| 124 | Snap in Place Button Release |
| 140 | Head Gear for Mask |
| 141 | Velcro ® for Straps |
| 150 | Human Head |

100—The CPAP mask system allows the user to roll over without the obstructive protrusion of a front mounted air inlet. Air inters from the right and left air inlet low profile channels on either side of your face. This low profile design allows the mask to stay in place while turning over in your sleep, and to feel a lot more comfortable. Dual valves and either side allow the user to exhale while in use.

110—Air flows into the air inlet and distribution chamber. This has a 360 degree snap fit hose connection. Allowing the user to turn in any direction without getting tangled in air hose. The chamber may have a bottom contoured to fit the top of a human head. A soft material or cushion may be attached to the bottom surface such that it is comfortable on the head. Velcro® or another attachment means may attach the chamber to the head gear to keep it in place.

120, 130—Low profile right & left air channels conform to the face because of soft low density plastic used to construct them. The air channels may have a flexible portion in the channel allowing them to stretch and fit your face and to help keep the mask in a comfortable position on your face. They also have a strap with Velcro® to help keep the desired length and fit. The ends of the low profile channels may have a push button snap fit and release.

The system's low profile CPAP mask may allow the user to turn 360 degrees without breaking the air tight seal of the mask. It may also keep the user from getting in tangled in air hoses.

The user may have a longer more comfortable night's sleep because the CPAP mask system allows a continuous, positive, air, pressure.

I claim:

1. A continuous positive air pressure mask system, configured to fit around a human head, comprising:
a continuous positive air pressure mask configured to fit over at least one of a nose and a mouth on the head, the mask including a first mask side and a second mask side opposite and mirroring the first mask side, the first mask side including a first air channel inlet and a first head gear attachment, the second mask side including a second air channel inlet and a second head gear attachment;
an air inlet distribution chamber including an air inlet chamber, a first air distribution outlet, a second air distribution outlet, and a chamber air inlet configured to connect to a continuous positive air pressure machine to receive pressurized air, the air inlet chamber including a bottom surface contoured to fit the top of the head;
a first side air channel fluidly connected to the mask at the first air channel inlet and the air inlet distribution chamber at the first air distribution outlet, and configured to be positioned on a first side of the head;
a second side air channel fluidly connected to the mask at the second air channel inlet and the air inlet distribution chamber at the second air distribution outlet to provide pressurized air to the mask, and configured to be positioned on a second side of the head; and
head gear releasably attached to the mask at the first head gear attachment and the second head gear attachment, and configured to hold the mask over the at least one of a nose and a mouth.
